(19) United States
(12) Patent Application Publication
(10) Pub. No.: US 2024/0033408 A1
(43) Pub. Date: Feb. 1, 2024

(54) EXTRACORPOREAL LIFE SUPPORT SYSTEM WITH BLOOD RECIRCULATION PATHWAY

(71) Applicant: CardiacAssist, Inc., Pittsburgh, PA (US)

(72) Inventors: Michael J. Linehan, Narberth, PA (US); Robert G. Svitek, Freeport, PA (US); Patrick A. Kelly, North Huntingdon, PA (US)

(73) Assignee: CardiacAssist, Inc., Pittsburgh, PA (US)

(21) Appl. No.: 17/876,012

(22) Filed: Jul. 28, 2022

(51) Int. Cl. A61M 1/16; A61B 5/145

(52) U.S. Cl. CPC A61M 1/1698; A61B 5/14542; A61M 2205/3334; A61M 2202/0208

(57) ABSTRACT

An extracorporeal blood treatment system including a blood oxygenator having an inlet for receiving deoxygenated blood and an outlet for expelling oxygenated blood. The system also includes a recirculation flow path for recirculating a portion of the oxygenated blood exiting the oxygenator outlet back into the oxygenator inlet. The system may also include a dual-lumen cannula coupled to the oxygenator. The cannula includes a manifold with a first blood pathway communicating with the oxygenator outlet, a second blood pathway communicating with the oxygenator inlet, and a third blood pathway connecting the first blood pathway to the second blood pathway. The manifold passes oxygenated blood received from the oxygenator through the first blood pathway, passes deoxygenated blood received from the patient through the second blood pathway, and passes a portion of the oxygenated blood from the first blood pathway through the third blood pathway to be combined with deoxygenated blood in the second blood pathway.

### TECHNICAL FIELD

The present disclosure relates to an extracorporeal life support system and methods for manufacturing and/or using an extracorporeal life support system. More particularly, the present disclosure pertains to extracorporeal life support systems that include a blood feedback line.

### BACKGROUND

Some medical procedures (e.g., medical procedures which treat cardiac or respiratory disease) may require the use of a life support system that supports cardiac and pulmonary functions by artificially supporting the heart and the lung function. In some instances, this may be carried out by an extracorporeal membrane oxygenation (ECMO) system, also known as an extracorporeal life support (ECLS) system. ECMO is an extracorporeal system which provides both cardiac and respiratory support to a patient whose heart and lungs are unable to provide an adequate amount of gas exchange to sustain life. ECMO works by removing blood from a patient's body to purify and oxygenate the red blood cells while also removing carbon dioxide. The purified and oxygenated blood is then returned to the patient.

ECMO systems may include multiple devices that together form a blood recirculation loop between the patient and a blood oxygenator. For example, some ECMO systems may include a blood reservoir, a blood pump to power blood flow, an oxygenator to oxygenate the blood, a device to filter the blood (which may be included within the oxygenator in some systems), a heat exchanger (to heat and/or cool blood), one or more oxygen sensors positioned at various locations along blood pathways and a control console. It can be appreciated that a blood pathway (e.g., tubing) may extend from the patient to the blood reservoir, then towards a blood pump, then pass through the oxygenator and close the loop by returning to the patient. Accordingly, the blood pump may assist the heart by pumping blood through the circulation loop, while the oxygenator may assist the lungs by oxygenating blood that is eventually returned to the patient.

It can be further appreciated that the amount of oxygen that can be delivered to the patient may be a function of the flow rate of the blood cycling through the circulation loop. However, there may be instances in which the flowrate of blood being taken from and returned to the patient may be capped and/or limited, such that additional oxygenation cannot be achieved by increasing the flowrate. Therefore, it may be desirable to design an ECMO system which can maximize the oxygen content in the blood returning to the patient without increasing the flowrate through the system. One method to maximize the oxygen content in the blood returning to the patient without increasing the flowrate may include adding a blood feedback line (e.g., a recirculation line) which feeds a portion of the oxygenated blood leaving the oxygenator back into the oxygenator for additional oxygenation. ECMO systems including a blood feedback line which feeds a portion of the oxygenated blood back into the oxygenator for additional oxygenation are disclosed herein.

### SUMMARY

An example extracorporeal blood treatment system may comprise a blood oxygenator having an inlet and an outlet. Deoxygenated blood received from a patient passes into the oxygenator inlet and oxygenated blood from the oxygenator exits through the outlet and passes to the patient. The system also includes a recirculation flow path configured to recirculate a portion of the oxygenated blood exiting the oxygenator outlet back into the oxygenator inlet.

In addition or alternatively to any example described herein, the recirculated oxygenated blood combines with the deoxygenated blood prior to passing through the oxygenator.

In addition or alternatively to any example described herein, the recirculated oxygenated blood combines with the deoxygenated blood within the oxygenator.

In addition or alternatively to any example described herein, the recirculated oxygenated blood combines with the deoxygenated blood within the recirculation flow path.

In addition or alternatively to any example described herein, the recirculated oxygenated blood has an oxygen saturation level, the deoxygenated blood has an oxygen saturation level, and combining the recirculated blood with the deoxygenated blood forms partially oxygenated blood having an oxygen saturation level which is between the oxygen saturation level of the oxygenated blood and the oxygen saturation level of the deoxygenated blood prior to passing through the oxygenator.

In addition or alternatively to any example described herein, the oxygen saturation of the partially oxygenated blood increases as the partially oxygenated blood passes through the oxygenator.

In addition or alternatively to any example described herein, the partially oxygenated blood passing through the oxygenator has a flowrate greater than the oxygenated blood exiting the oxygenator and returning to the patient.

In addition or alternatively to any example described herein, the partially oxygenated blood passing through the oxygenator has a flowrate equal to the flowrate of the blood exiting the oxygenator and returning to the patient plus the flowrate of the oxygenated blood passing into the recirculation flow path.

In addition or alternatively to any example described herein, the system further comprises a recirculation pump coupled to the oxygenator, wherein the recirculation pump is configured to pump the recirculated oxygenated blood back into the oxygenator

In addition or alternatively to any example described herein, the system further comprises a blood pump coupled to the oxygenator, wherein the blood pump is configured to pump deoxygenated blood from the patient into the oxygenator In addition or alternatively to any example described herein, blood passes from the oxygenator to the patient along a first blood pathway, and the first blood pathway includes a first oxygen sensor positioned therein.

In addition or alternatively to any example described herein, blood passes from the patient to the oxygenator along a second blood pathway, and the second blood pathway includes a second oxygen sensor positioned therein.

In addition or alternatively to any example described herein, the first oxygen sensor is configured to sense an oxygen saturation level of blood in the first blood pathway, the second oxygen sensor is configured to sense an oxygen saturation level of blood in the second blood pathway, and the first oxygen sensor, the second oxygen sensor or both the first oxygen sensor and the second oxygen sensor are configured to send a signal to the oxygenator indicating the oxygen saturation level of blood in the first blood pathway and the second blood pathway, respectively.

In addition or alternatively to any example described herein, the oxygenator is configurated to adjust the oxygen saturation level of blood in the first blood pathway in response to a signal received from the first oxygen sensor, the second oxygen sensor or both the first oxygen sensor and the second oxygen sensor.

In addition or alternatively to any example described herein, the system further includes a dual lumen cannula coupled to the oxygenator, the dual lumen cannula including a manifold having a first blood pathway, a second blood pathway and a third blood pathway, wherein the third blood pathway connects the first blood pathway to the second blood pathway.

In addition or alternatively to any example described herein, the oxygenated blood passes from the oxygenator to the patient through the first blood pathway of the manifold, deoxygenated blood passes from the patient to the oxygenator through the second blood pathway of the manifold, and a portion of the oxygenated blood passes from the first blood pathway, through the third blood pathway and combines with deoxygenated blood in the second blood pathway.

Another illustrative example is an extracorporeal blood treatment system, comprising a blood circulation pathway coupled to a blood oxygenator and a blood recirculation pathway. The blood circulation pathway is configured to pass deoxygenated blood withdrawn from a patient through a blood oxygenator and back to the patient. The blood recirculation pathway is configured to recirculate oxygenated blood exiting the oxygenator back into the oxygenator prior to returning to the patient.

In addition or alternatively to any example described herein, the blood passing through oxygenator has a flowrate greater than the oxygenated blood exiting the oxygenator and passing to the patient.

In addition or alternatively to any example described herein, the blood passing through the oxygenator has a flowrate equal to the flowrate of the oxygenated blood exiting the oxygenator and passing to the patient plus the flowrate of oxygenated blood exiting the oxygenator and passing into the blood recirculation pathway.

Yet another illustrative example is an extracorporeal blood treatment system, comprising a blood oxygenator having an inlet and an outlet, and a dual-lumen cannula coupled to the oxygenator. The cannula having a distal end configured to be positioned in a patient and a proximal end including a manifold. The manifold includes a first blood pathway in fluid communication with the oxygenator outlet, a second blood pathway in fluid communication with the oxygenator inlet, and a third blood pathway connecting the first blood pathway to the second blood pathway. The manifold is configured to pass oxygenated blood received from the oxygenator through the first blood pathway. The manifold is configured to pass deoxygenated blood received from the patient through the second blood pathway. The manifold is configured to pass a portion of the oxygenated blood from the first blood pathway through the third blood pathway such that it combines with deoxygenated blood in the second blood pathway.

The above summary of some embodiments, aspects, and/or examples is not intended to describe each disclosed embodiment or every implementation of the present disclosure. The figures and detailed description which follow more particularly exemplify these embodiments.

### BRIEF DESCRIPTION OF THE DRAWINGS

The disclosure may be more completely understood in consideration of the following detailed description in connection with the accompanying drawings, in which:

FIG. 1A illustrates an example extracorporeal blood treatment system;

FIG. 1B illustrates another example extracorporeal blood treatment system;

FIG. 2A illustrates another example extracorporeal blood treatment system;

FIG. 2B illustrates another example extracorporeal blood treatment system;

FIG. 3 illustrates an example extracorporeal blood treatment system including a dual-lumen cannula;

FIG. 4 illustrates an example manifold of the dual-lumen cannula of FIG. 3.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5). As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used in connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the disclosure.

In a normal heart, blood circulates via a closed path whereby deoxygenated (venous) blood enters the right atrium via both the superior vena cava and inferior vena cava. The venous blood then passes through the right ventricle and is pumped via the pulmonary artery to the lungs, where it absorbs oxygen. After absorbing oxygen in the lungs, the blood becomes oxygenated arterial blood. The oxygenated arterial blood is then returned via the pulmonary veins to the left atrium and is passed to the left ventricle. The oxygenated arterial blood is then pumped through the aorta and eventually throughout the body.

It can be appreciated that if the lungs are incapable of sufficiently oxygenating blood, an oxygenator located outside the body may be used to oxygenate the blood. As discussed above, extracorporeal membrane oxygenation (ECMO) is a breathing and heart pumping life support system that may be utilized to support patients while medical treatments are performed to treat their underlying illness. When supported via an ECMO system, oxygenation of the patient's blood and removal of carbon dioxide may occur outside the body.

ECMO is generally performed using a heart-lung bypass system, which may be referred to as a “circuit.” The circuit may include one or more tubing pathways designed to transfer blood from a patient's body to the oxygenator and back into the patient. As described above, the oxygenator may add oxygen to the blood while also removing carbon dioxide (e.g., the oxygenator performs the function of a healthy lung).

In some examples, an ECMO circuit may include a blood pump, oxygenator, tubing pathways (for transfer to and from the body), flow and/or pressure sensors, a heat exchanger (to cool and/or heat the blood), a computing console, and arterial and/or venous access points for the collection of blood in the circuit. It can be appreciated that the function of the blood pump is to generate blood flow within the ECMO circuit (e.g., circulate blood from the patient to the oxygenator and back to the patient). The blood pump may be positioned in the tubing pathway between the patient and the oxygenator. In some ECMO systems a roller pump may be utilized to generate blood flow within the ECMO circuit. However, in other ECMO systems, other blood pumps, including centrifugal pumps may be utilized to generate blood flow within the ECMO circuit.

In some ECMO systems, the oxygenator may include a housing having multiple chambers or pathways separated by a semi-permeable membrane, whereby the patient's blood may flow through one chamber or pathway, while an oxygen gas mixture (i.e., sweep gas) flows through another chamber or pathway. The semi-permeable membrane may include multiple microporous hollow fibers, each fiber having lumen extending therethrough through which the oxygen gas mixture flows. The gas exchange may occur via diffusion of the gases across multiple microporous fibers, whereby oxygen moves from the inside of the hollow fibers into the blood while carbon dioxide diffuses from the blood into the interior of the hollow fibers, where it is swept away by the sweep gas flowing through the fiber. This gas exchange allows for oxygenation of venous blood and removal of carbon dioxide. In some ECMO systems, the oxygenator may include integrated heat exchangers that allow circulating blood to be cooled and/or warmed prior to returning to the patient.

It can be appreciated that the saturation level of oxygen in the blood after passing through the oxygenator membrane (e.g., post-oxygenated blood) may be a function of the volume or flowrate of oxygen gas flowing through the semi-permeable membrane (e.g., hollow fibers) and also the flowrate of blood passing across the semi-permeable membrane (e.g., hollow fibers). Accordingly, an increase in the volume or flowrate of blood across the semi-permeable membrane may increase the volume of oxygen delivered in the post-oxygenated blood. As discussed above, there may be instances in which the flowrate of blood within the ECMO circuit may be capped and/or limited, such that additional oxygenation cannot be achieved by increasing the flowrate of blood through the oxygenator. One method to maximize the oxygen content in the blood returning to the patient without increasing the flowrate of the system to and from the patient may include adding a blood feedback line which feeds a portion of the oxygenated blood back into the oxygenator for additional oxygenation, thus increasing the flowrate of blood through the oxygenator without increasing the flowrate of blood to/from the patient.

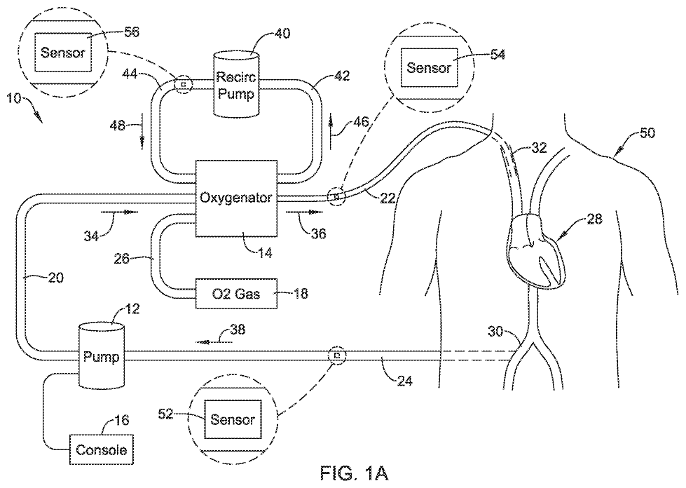

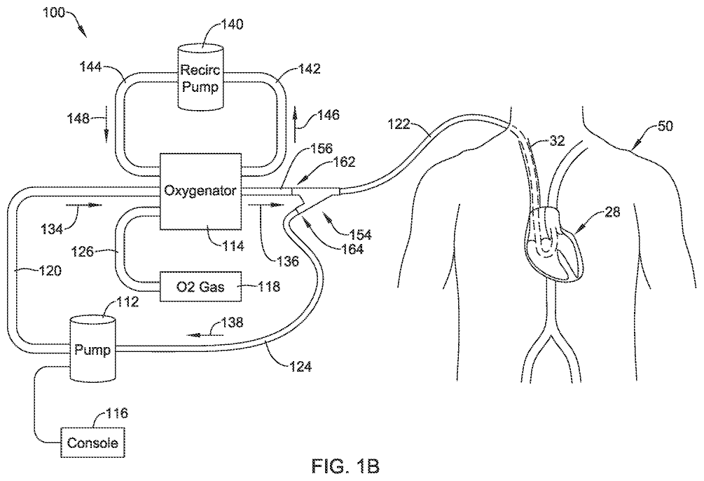

FIG. 1A illustrates an ECMO system 10 including a blood feedback line which feeds a portion of oxygenated blood back to the oxygenator for additional oxygenation. The ECMO system 10 may include a blood pump 12 designed to draw deoxygenated blood from a patient 50 and propel the blood to an oxygenator 14. Further, after the blood passes through the semi-permeable membrane of the oxygenator 14, the post-oxygenated blood may return to the patient 50.

FIG. 1A illustrates a type of ECMO life support system designed to support lung function. This type of ECMO system may be referred to as a veno-venous (VV) ECMO life support system. The veno-venous ECMO system shown in FIG. 1A may include two separate cannulation sites (e.g., sites at which tubular members are inserted into the patient's body). For example, FIG. 1A shows a first cannulation site in which a cannula is inserted into the femoral vein 30. While not illustrated in FIG. 1A, the cannula inserted into the femoral vein 30 may be tracked into the inferior vena cava, for example, whereby it may be utilized to drain deoxygenated blood from the patient. As illustrated in FIG. 1A, a cannula inserted into the femoral vein 30 may be connected to a tubular pathway 24 (e.g., a length of tubing) which may be coupled to the pump 12. Therefore, it can be appreciated that the pump 12 may be utilized to remove (e.g., pull, drain, draw, etc.) deoxygenated blood from the patient 50.

FIG. 1A further illustrates that the ECMO life support system may further include a tubular pathway 20 (e.g., a length of tubing) extending between the pump 12 and the oxygenator 14. The tubular pathway 20 may be an extension of the tubular pathway 24. For example, the tubular pathway 20 and the tubular pathway 24 may be a continuous tubular member which passes through and engages with the pump 12. Alternatively, the tubular pathway 20 and the tubular pathway 24 may be separate tubular members, each of which include an end region that connects to the pump 12. In other instances, the pump 12 may be directly attached to or integral with the oxygenator 14, thus eliminating the need for the tubular pathway 20 therebetween. The tubular pathways 20/24 may be referred to as a drainage line removing deoxygenated blood from the patient.

Additionally, the veno-venous ECMO system shown in FIG. 1A shows a second cannulation site in which a cannula is inserted into the right jugular vein 32. While not illustrated in FIG. 1A, the cannula inserted into the right jugular vein 32 may be tracked into the right atrium, for example, whereby it may be utilized to return oxygenated blood to the patient. As illustrated in FIG. 1A, a cannula inserted into the right jugular vein 32 may be connected to a tubular pathway 22 (e.g., a length of tubing) which may be coupled to the oxygenator 14. It can be appreciated that the pump 12 may be utilized to propel oxygenated blood from the oxygenator 14 to the patient 50. The tubular pathway 22 may be referred to as a return line returning oxygenated blood back to the patient.

FIG. 1A further illustrates that the ECMO life support system 10 may further include an oxygen source 18 coupled to the oxygenator 14. The oxygen source 18, in some instances, may include an oxygen tank which is coupled to the oxygenator 14 via the tubular pathway 26. The oxygenator 14 may draw oxygen from the oxygen source 18 to oxygenate blood passing through the semi-permeable membrane of the oxygenator 14.

As discussed herein, the ECMO system 10 may include a blood pump 12 designed to draw deoxygenated blood from the inferior vena cava of a patient 50 (the direction of deoxygenated blood out of the patient is shown by arrow 38) and propel the blood to an oxygenator 14 (the direction of deoxygenated blood from the pump to the oxygenator is shown by arrow 34). After the deoxygenated blood enters the oxygenator 14 it may pass through the semi-permeable membrane of the oxygenator 14 whereby red blood cells absorb oxygen (drawn from the oxygen source 18) and carbon dioxide is released. After blood passes through the semi-permeable membrane of the oxygenator 14, the post-oxygenated blood may return to the patient (the direction of post-oxygenated blood going to the patient is shown by arrow 36) through the tubular pathway 22. After passing through a cannula inserted into the right jugular vein 32, the post-oxygenated blood may be released in the right atrium of the patient 50.

As discussed herein, deoxygenated blood may enter the oxygenator 14 whereby it may pass through or by the semi-permeable membrane of the oxygenator 14 to absorb oxygen. In general, the term “deoxygenated blood” may be defined as blood that has a low oxygen saturation relative to blood leaving the lungs. For example, in a normal patient, the oxygen saturation level of oxygenated blood leaving lungs may be 95% or higher. Accordingly, it can be appreciated that the oxygen saturation level of deoxygenated blood leaving the patient 50 and flowing into the ECMO system 10 of FIG. 1A may be approximately 65%. Further, if the deoxygenated blood leaving the patient passes through the oxygenator 14 at a constant flowrate and constant volume, the oxygen saturation level in the post-oxygenated blood may increase to 95% or higher.

It can be further appreciated that if the ECMO system 10 shown in FIG. 1A is operated at a constant flowrate, the oxygen saturation level in the post-oxygenated blood may remain at a substantially constant level. However, in some instances it may be desirable to increase the blood saturation level of the post-oxygenated blood. As discussed herein, one method to increase the blood saturation level of the post-oxygenated blood may be to increase the flowrate of the blood through the oxygenator 14. However, if a patient lacks enough blood volume to increase the blood flowrate sufficiently, then increasing blood flowrate may cause vessel walls to collapse on the drainage and/or return cannulas, thereby preventing blood flow entirely. Therefore, it may be desirable to increase the blood saturation level of the post-oxygenated blood without increasing the flowrate of the blood in the system to/from the patient.

The system illustrated in FIG. 1A may increase the blood saturation level of the post-oxygenated blood without increasing the flowrate of the blood in the system to/from the patient. Specifically, FIG. 1A illustrates that the ECMO system 10 may include a blood feedback line which transfers post-oxygenated blood from the outlet of the oxygenator 14 and returns it back to the inlet of the oxygenator 14. The blood in the feedback line may combine with deoxygenated blood being drawn from the patient 50 to be passed through the oxygenator 14. For example, FIG. 1A illustrates a recirculation pump 40 which may be designed to direct (e.g., pull, drain, draw, etc.) post-oxygenated blood output from the oxygenator 14 through a tubular pathway 42 (the direction of post-oxygenated blood flowing out of the oxygenator 14 through the recirculation pathway is shown by arrow 46). Further, FIG. 1A illustrates the “recirculated” post-oxygenated blood passing through the recirculation pump 40 and returning to the oxygenator 14 via the tubular pathway 44 (the direction of post-oxygenated blood flowing out of the recirculation pump 40 and returning to the oxygenator 14 is shown by arrow 48).

It can be appreciated that as the post-oxygenated blood enters the oxygenator 14 from the recirculation pathway 42/44, it may combine with the deoxygenated blood from the drainage pathway from the patient prior to passing through or by the semi-permeable membrane of the oxygenator 14. Accordingly, the recirculated post-oxygenated blood, which is mixed with the deoxygenated blood, may increase the oxygen level in the deoxygenated blood coming from the drainage pathway from the patient before it passes through the semi-permeable membrane of the oxygenator 14. The oxygen level in the deoxygenated blood that has been combined with the recirculated post-oxygenated blood may have an oxygen saturation level that is between the oxygen saturation level of the deoxygenated blood and the oxygen saturation level of the post-oxygenated blood passing through the recirculation pathway. Thus, the mixture of blood entering the oxygenator 14 may have an oxygen saturation level that is between the oxygen saturation level of the deoxygenated blood and the oxygen saturation level of the post-oxygenated blood passing through the recirculation pathway. After the deoxygenated blood is combined with the recirculated post-oxygenated blood, the mixture of blood may then pass through the oxygenator 14, whereby its oxygen level is further increased. It can be further appreciated that as this “recirculation cycle” continues (e.g., as a portion of the post-oxygenated blood is recirculated back into the oxygenator 14 to mix with deoxygenated blood coming from the patient), the oxygen saturation level in the post-oxygenated blood exiting the oxygenator 14 may increase over time to 100% saturation. Additionally, once the hemoglobin is saturated to 100%, additional oxygen will be added in the form of dissolved oxygen into the plasma which can add approximately 10% more oxygen content to the blood.

Additionally, it can be appreciated that the flowrate of the blood passing through the drainage pathway from the patient to the oxygenator 14 (via tubular pathways 24/20), the flowrate of the post-oxygenated blood passing within the recirculation flow path (via tubular pathways 42/44), and the flowrate of the post-oxygenated blood returning to the patient (via the tubular flow pathway 22) may have different values. Specifically, the flowrate of the blood passing through the oxygenator 14 may be equal to the sum of the flowrate of the blood passing through the recirculation flow path and the flowrate of the blood coming from/returning to the patient. Therefore, the flowrate of the blood coming from the patient and the flowrate of blood returning to the patient may be less than the flowrate of the blood passing through the oxygenator 14. Further, the flowrate of the blood passing through the recirculation flow path is also less than the flowrate of the blood passing through the oxygenator 14.

Additionally, FIG. 1A illustrates that in some examples, the ECMO system 10 may include a controller or console 16 coupled to various components of the ECMO system 10. For example, FIG. 1A illustrates that the console 16 may be coupled to the pump 12. However, it is also contemplated that the console 16 may be coupled to the oxygenator 14, the recirculation pump 40 and/or the oxygen source 18. Further, the console 16 may include a computing device. The console 16 may further include, among other suitable components, a processor, display, memory, and an I/O unit.

The processor of the console 16 may include a single processor or more than one processor working individually or with one another. The processor may be configured to execute instructions, including instructions that may be loaded into the memory and/or other suitable memory. Example processor components may include, but are not limited to, microprocessors, microcontrollers, multi-core processors, graphical processing units, digital signal processors, application specific integrated circuits (ASICs), field programmable gate arrays (FPGAs), discrete circuitry, and/or other suitable types of data processing devices.

The memory of the console 16 may include a single memory component or more than one memory component each working individually or with one another. Example types of memory may include random access memory (RAM), EEPROM, FLASH, suitable volatile storage devices, suitable non-volatile storage devices, persistent memory (e.g., read only memory (ROM), hard drive, Flash memory, optical disc memory, and/or other suitable persistent memory) and/or other suitable types of memory. The memory may be or may include a non-transitory computer readable medium.

The I/O units of the console 16 may include a single I/O component or more than one I/O component each working individually or with one another. Example I/O units may be any type of communication port configured to communicate with other components of the building management system. Example types of I/O units may include wired ports, wireless ports, radio frequency (RF) ports, Low-Energy Bluetooth ports, Bluetooth ports, Near-Field Communication (NFC) ports, HDMI ports, WiFi ports, Ethernet ports, VGA ports, serial ports, parallel ports, component video ports, S-video ports, composite audio/video ports, DVI ports, USB ports, optical ports, and/or other suitable ports.

Additionally, FIG. 1A illustrates that the ECMO system 10 may include one or more sensors 52/54/56 positioned in one or more tubular pathways connecting various components of the ECMO system 10. For example, FIG. 1A illustrates a sensor 52 positioned in the tubular pathway 24, a sensor 54 positioned in the tubular pathway 22 and a sensor 56 positioned in the tubular pathway 44. In some examples, the sensors 52/54/56 may be oxygen sensors, carbon dioxide sensors, pressure sensors, flowrate sensors, or the like. Additionally, it can be appreciated that the console 16 may be designed to communicate with one or more of the sensors 52/54/56. For example, the sensors 52/54/56 may be oxygen sensors designed to sense the oxygen saturation levels in the blood flowing through tubular pathways 24/22/44, respectively. For example, the sensor 52 may sense the oxygen saturation level and/or flowrate of deoxygenated blood being removed from the patient, the sensor 54 may sense the oxygen saturation level and/or flowrate of oxygenated blood being returned to the patient and the sensor 44 may sense the oxygen saturation level and/or flowrate of oxygenated blood in the recirculation flowpath. In some examples, the values sensed by the sensors 52/54/56 may be transmitted (e.g., communicated) back to the console 16. The values (e.g., data) transmitted back to the console may be utilized to calculate and control flowrates for additional oxygenation. Transmission of the sensed values may be performed via a wireless connection, a wired connection or other communication means capable of transmitting signals between the console 16 and the sensors 52/54/56.

Additionally, it can be appreciated that the console 16 may be communicate with and adjust various components of the ECMO system 10 in response to sensed signals sent from the sensors 52/54/56 to the console 16. For example, the sensor 52 may sense and transmit one or more oxygen saturation levels of the blood in the tubular pathway 24, the sensor 54 may sense and transmit one or more oxygen saturation levels of the blood in the tubular pathway 22 and the sensor 56 may sense and transmit one or more oxygen saturation levels of the blood in the tubular pathway 44. Based on the received signals, the console 16 may communicate with various components to adjust the oxygen saturation levels in the ECMO circuit in the oxygenated blood being returned to the patient. For example, the console 16 may communicate with the recirculation pump 40, whereby the console 16 increases the pumping action of the recirculation pump 40 (e.g., increases the rotational speed of the recirculation pump 40) to increase flowrate of blood within the recirculation circuit. The increased flowrate of the recirculation pump 40 may increase the amount of blood passing through the oxygenator 14, thereby increasing the oxygen saturation level in the post-oxygenated blood. Furthermore, the console 16 may communicate with the recirculation pump 40, whereby the console 16 decreases the pumping action of the recirculation pump 40 (e.g., decreases the rotational speed of the recirculation pump 40) to decrease flowrate of blood within the recirculation circuit. The decreased flowrate of the recirculation pump 40 may decrease the amount of blood passing through the oxygenator 14, thereby reducing the oxygen saturation level in the post-oxygenated blood. In other instances, the console 16 may communicate with an adjustable flow restrictor (not shown) placed in the recirculation circuit (e.g., along flow path 42/44) to adjust the flowrate of blood within the recirculation circuit.

It can be appreciated that other parameters may be sensed and communicated to the console 16, whereby the console 16 adjusts one or more components of the ECMO circuit based on the sensed parameters. For example, the sensors 52/54/56 may sense the flowrate of blood within one or more tubular pathways (e.g., the tubular pathways 20/22/24/42/44) of the ECMO circuit and communicate the flowrate(s) to the console 16. Based on the sensed flowrates, the console 16 may communicate with various components of the EMCO system 10 to adjust the various components in the ECMO circuit. For example, the console 16 may communicate with the pump 12, whereby the console 16 adjusts (e.g., increases or decreases) the pumping action of the pump 12 (e.g., increases or decreases the rotational speed of the pump 12) to increase the flowrate of blood within the tubular pathways 22/24/44. The increased flowrate of the pump 12 may increase the amount of blood passing through the oxygenator 14, thereby increasing the oxygen saturation level in the post-oxygenated blood. Decreasing the flowrate of the pump 12 may decrease the amount of blood passing through the oxygenator 14, thereby decreasing the oxygen saturation level in the post-oxygenated blood. It can be appreciated that form and functionality of the console 116 and the sensors 52/54/56 described herein may be applied to any of the ECMO life support systems disclosed with respect to FIGS. 1B-5.

While the above discussion describes a veno-venous ECMO life support system utilized with a recirculation circuit, it is contemplated that a recirculation circuit (such as that described with respect to FIG. 1A) may be utilized with different ECMO life support systems. For example, a recirculation circuit (such as that described with respect to FIG. 1A) may be utilized with a veno-atrial ECMO life support system. It can be appreciated that veno-venous and veno-atrial ECMO life support systems may have a variety of cannulation configurations.

For example, FIG. 1B illustrates another veno-venous ECMO life support system 100. The ECMO system 100 is similar in form and function to the ECMO system 10 illustrated in FIG. 1A. For example, the ECMO system 10 may include a blood pump 112 designed to remove deoxygenated blood from a patient 50 through a tubular blood pathway 124 (the direction of deoxygenated blood out of the patient is shown by arrow 138) and propel the blood to an oxygenator 114 through a tubular blood pathway 120 (the direction of deoxygenated blood from the pump to the oxygenator is shown by arrow 134). After the deoxygenated blood enters the oxygenator 114 it may pass through the semi-permeable membrane of the oxygenator 114 whereby red blood cells absorb oxygen (drawn from an oxygen source 118) and carbon dioxide is released.

Further, after blood passes through the semi-permeable membrane of the oxygenator 114, some of the post-oxygenated blood may return to the oxygenator 114 via a recirculation pathway whereby a recirculation pump 140 may direct (e.g., pull, drain, draw, etc.) post-oxygenated blood out of the oxygenator 114 through a tubular pathway 142 (the direction of post-oxygenated blood flowing out of the oxygenator 114 is shown by arrow 146). Further, the recirculated post-oxygenated blood may pass through the recirculation pump 140 and return to the oxygenator 114 via the tubular pathway 144 (the direction of post-oxygenated blood flowing out of the oxygenator 114 is shown by arrow 148).

Similar to the feedback pathway described above with respect to FIG. 1A, the post-oxygenated blood that enters the oxygenator 114 may combine with the deoxygenated blood prior to passing through the semi-permeable membrane of the oxygenator 114. Accordingly, the recirculated post-oxygenated blood may increase the oxygen level in the deoxygenated blood before it passes through the semi-permeable membrane of the oxygenator 114. It can be further appreciated that as this recirculation cycle continues (e.g., as post-oxygenated blood is recirculated back into the oxygenator 114 to mix with deoxygenated blood), the oxygen saturation level in the post-oxygenated blood leaving the oxygenator 114 will increase over time.

However, alternatively to the dual cannulation site ECMO system 10 described with respect to FIG. 1A, the ECMO system 110 shown in FIG. 1B utilizes a dual-lumen cannula 122 introduced into the patient 50 via the internal jugular vein 32 whereby it is further tracked into the right atrium of the heart. The dual lumen cannula 122 may include a first, infusion lumen (e.g., inner lumen) extending coaxially within a second, drainage lumen (e.g., outer lumen). Accordingly, fluid (e.g., blood) may flow through both the infusion lumen and the drainage lumen. While not illustrated in FIG. 1B, the drainage lumen may include one or more apertures disposed along its distal end region in fluid communication with the drainage lumen. Additionally, the infusion lumen may extend distally past the end of the drainage lumen. When positioned in the heart, the drainage lumen is positioned in the right atrium and infusion lumen is positioned in the main pulmonary artery, for example.

The dual-lumen cannula 122 operates by returning oxygenated blood to the patient from the ECMO circuit via its infusion lumen, which may be positioned in the pulmonary artery, and withdrawing deoxygenated blood from the patient to the ECMO circuit via its drainage lumen, which may be positioned in the right atrium. FIG. 1B illustrates that the dual-lumen cannula 122 may include a hub or manifold 154 having an inlet port 162 and an outlet port 164. It can be appreciated that the inlet port 162 may be coupled to an outlet of the oxygenator 114. Accordingly, post-oxygenated blood exiting the oxygenator 114 may enter the manifold inlet port 162, whereby the post-oxygenated blood passes through the infusion lumen and into the main pulmonary artery. Further, it can be appreciated that the outlet port 164 of the manifold 154 may be coupled to the tubular blood pathway 124. Accordingly, deoxygenated blood may drain through the drainage lumen positioned in the right atrium, travel through the drainage lumen and pass into the tubular blood pathway 124, whereby the pump 112 may propel it toward the oxygenator 114. After passing through the oxygenator 114 and through the infusion lumen of the dual-lumen cannula 122 inserted into the right jugular vein 32, the post-oxygenated blood may be released in the right atrium of the patient 50.

Similar to the ECMO system 10 discussed above, the ECMO system 100 may include one or more sensors (such as sensors 52/54/56) positioned in one or more tubular pathways connecting various components of the ECMO system 100. For example, a sensor may be positioned in the tubular pathway 124 and a sensor may be positioned in the tubular pathway 122. In some examples, the sensors may be oxygen sensors, flowrate sensors, or the like. Additionally, it can be appreciated that the console 16 may be designed to communicate with one or more of the sensors. For example, the sensors may be oxygen sensors designed to sense the oxygen saturation levels in the blood flowing through tubular pathways 124/122, respectively. For example, the sensor in the pathway 124 may sense the oxygen saturation level and/or flowrate of deoxygenated blood being removed from the patient, whereas the sensor in the pathway 122 may sense the oxygen saturation level and/or flowrate of oxygenated blood being returned to the patient. In some examples, the values sensed by the sensors may be transmitted (e.g., communicated) back to the console 116. Transmission of the sensed values may be performed via a wireless connection, a wired connection or other communication means capable of transmitting signals between the console 116 and the sensors. As discussed above, it can be appreciated that the console 116 may be communicate with and adjust various components of the ECMO system 100 in response to sensed signals sent from the sensors to the console 116.

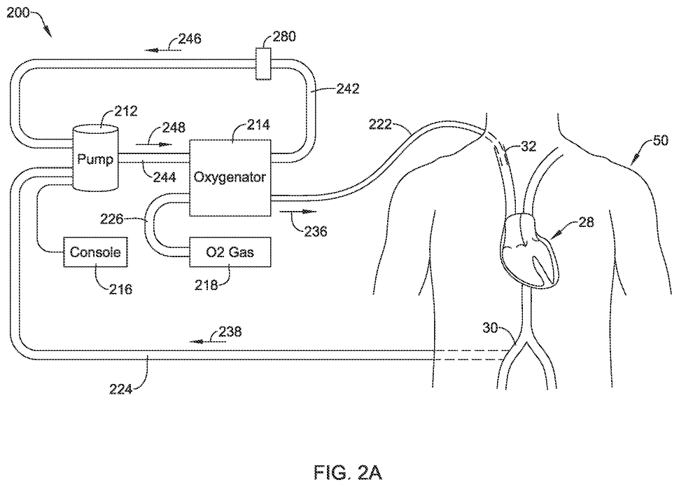

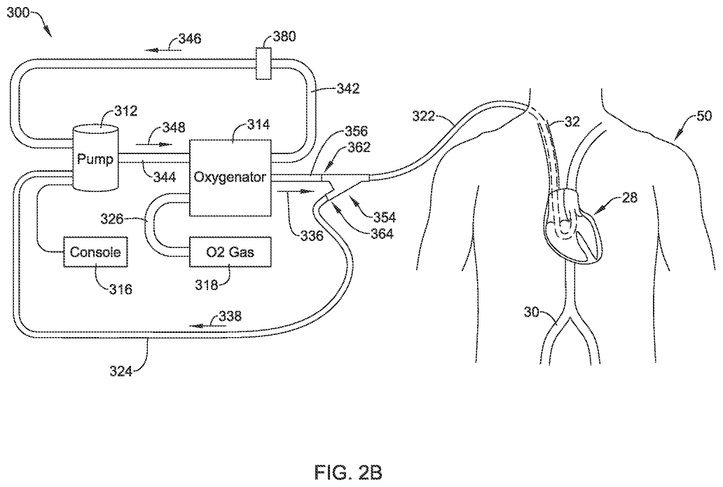

FIG. 2A illustrates another veno-venous ECMO life support system 200. The ECMO system 200 is similar in form and function to the EMCO system 10 illustrated in FIG. 1A. For example, FIG. 2A shows a first cannulation site in which a cannula is inserted into the femoral vein 30. While not illustrated in FIG. 2A, the cannula inserted into the femoral vein 30 may be tracked into the inferior vena cava, whereby it may be utilized to remove deoxygenated blood from the patient. As illustrated in FIG. 2A, a cannula inserted into the femoral vein 30 may be connected to a tubular pathway 224 (e.g., a length of tubing) which may be coupled to the pump 212. Therefore, it can be appreciated that the pump 212 may be utilized to remove (e.g., pull, drain, draw, etc.) deoxygenated blood from the patient 50 (the direction of deoxygenated blood out of the patient is shown by arrow 238).

It can be further appreciated that the pump 212 may also be utilized to propel blood to an oxygenator 214 through a tubular pathway 244 (the direction of deoxygenated blood flowing through the pump 212 toward the oxygenator 214 is shown by arrow 248). After blood passes by or through the semi-permeable membrane of the oxygenator 214, the post-oxygenated blood may return to the patient (the direction of post-oxygenated blood going to the patient is shown by arrow 236). After passing through a cannula inserted into the right jugular vein 32, the post-oxygenated blood may be released in the right atrium of the patient 50.

Further, the ECMO system 200 may include a recirculation circuit to recirculate blood through the oxygenator 214. As described above with respect to FIG. 1A, after blood passes by or through the semi-permeable membrane of the oxygenator 214, some of the post-oxygenated blood may return to the inflow side of the oxygenator 214 via a blood feedback line. However, alternatively to the design disclosed with respect to FIG. 1A, the ECMO system 200 shown in FIG. 2A may not include a separate recirculation pump dedicated to drive the post-oxygenated blood feedback pathway (e.g., blood flow through the tubular pathway 242). Rather, FIG. 2A illustrates that the ECMO system 200 may utilize the pump 212 to siphon (e.g., pull, drain, draw, etc.) post-oxygenated blood out of the oxygenator 214 through a recirculation tubular pathway 242 (the direction of post-oxygenated blood flowing out of the oxygenator 214 is shown by arrow 246).

As illustrated in FIG. 2A, the recirculated post-oxygenated blood may pass through the pump 212 and return to the inlet side of the oxygenator 214 via the tubular pathway 244 (the direction of post-oxygenated blood flowing out of the oxygenator 214 is shown by arrow 248). It can be appreciated the post-oxygenated blood that enters the pump 212 may combine with the deoxygenated blood being drawn from the patient through the tubular pathway 224 prior to passing by or through the semi-permeable membrane of the oxygenator 214. It is further contemplated that the post-oxygenated blood within the feedback pathway may combine with deoxygenated blood prior to entering the pump 212, within the pump 212 or within the tubular pathway 244. Accordingly, the recirculated post-oxygenated blood may increase the oxygen level in the deoxygenated blood before it passes through the semi-permeable membrane of the oxygenator 214. It can be further appreciated that as the oxygen recirculation cycle continues (e.g., as post-oxygenated blood is recirculated and mixed with deoxygenated blood prior to entering the oxygenator 214), the oxygen saturation level in the post-oxygenated blood returning to the patient will increase over time.

In the ECMO system 200, a flow regulator 280 may be positioned in the blood recirculation pathway 242 to regulate the flowrate of blood being recirculated through the pump 212 to the oxygenator 214. The flow regulator 280 may be controlled by the console 216, for example, to automatically adjust the orifice through the flow regulator 280 in response to a desired demand for adjusting the oxygen saturation level of oxygenated blood exiting the oxygenator 214 and/or returning to the patient 50. For instance, based on the oxygen saturation level of post-oxygenated blood returning to the patient, which may be sensed with an oxygen sensor as described above, the console 216 may automatically adjust (increase or decrease) the flow of blood through the flow regulator 280. In other instances, a user may manually adjust (increase or decrease) the flow of blood through the flow regulator 280 with the console or other controller.

FIG. 2B illustrates another veno-venous ECMO life support system 300. The ECMO system 300 may be similar in form and function to the EMCO system 100 illustrated in FIG. 1B. For example, the ECMO system 100 may include a blood pump 312 designed to remove deoxygenated blood from a patient 50 through a tubular blood pathway 324 (the direction of deoxygenated blood out of the patient is shown by arrow 338). It can be further appreciated that the pump 312 may also be utilized to propel blood to an oxygenator 314 through a tubular pathway 344 (the direction of deoxygenated blood flowing through the pump 312 toward the oxygenator 314 is shown by arrow 348). As the deoxygenated blood enters the oxygenator 114 it may pass by or through the semi-permeable membrane of the oxygenator 114 whereby red blood cells absorb oxygen (drawn from an oxygen source 118) and carbon dioxide is released. Further, after blood passes through the semi-permeable membrane of the oxygenator 314, the post-oxygenated blood may return to the patient (the direction of post-oxygenated blood exiting an outlet port of the oxygenator is shown by arrow 336).

Like the dual cannulation site ECMO system 100 described with respect to FIG. 1B, the ECMO system 300 shown in FIG. 2B utilizes a dual-lumen cannula 322 introduced into the patient 50 via the internal jugular vein 32 whereby it is further tracked into the right atrium of the heart. As described herein, the dual lumen cannula 322 may include a first, infusion lumen (e.g., inner lumen) extending coaxially within a second, drainage lumen (e.g., outer lumen). Accordingly, fluid (e.g., blood) may flow through both the infusion lumen and the drainage lumen. While not illustrated in FIG. 2B, the drainage lumen may include one or more apertures disposed along its distal end region in fluid communication with the drainage lumen. Additionally, the infusion lumen may extend distally past the end of the drainage lumen. When positioned in the heart, the drainage lumen is positioned in the right atrium and infusion lumen is positioned in the main pulmonary artery, for example.

As discussed herein, the dual-lumen cannula 322 operates by returning oxygenated blood to the patient from the ECMO circuit via its infusion lumen, which may be positioned in the pulmonary artery, and withdrawing deoxygenated blood from the patient to the ECMO circuit via its drainage lumen, which may be positioned in the right atrium. FIG. 2B illustrates that the dual-lumen cannula 322 may include a hub or manifold 354 having an inlet port 362 and an outlet port 364. It can be appreciated that the inlet port 362 may be coupled to an outlet of the oxygenator 314. Accordingly, post-oxygenated blood exiting the oxygenator 314 may enter the manifold inlet port 362, whereby the post-oxygenated blood passes through the infusion lumen and into the main pulmonary artery. Further, it can be appreciated that the outlet port 364 of the manifold 354 may be coupled to the tubular blood pathway 324. Accordingly, deoxygenated blood may drain through the drainage lumen positioned in the right atrium, travel through the drainage lumen and pass into the tubular blood pathway 324, whereby the pump 312 may propel it toward the oxygenator 314. After passing through the oxygenator 314 and through the infusion lumen of the dual-lumen cannula 322 inserted into the right jugular vein 32, the post-oxygenated blood may be released in the right atrium of the patient 50.

Further, the ECMO system 300 may include a recirculation or feedback circuit to recirculate blood through the oxygenator 314. As described above with respect to FIG. 1B, after blood passes by or through the semi-permeable membrane of the oxygenator 314, some of the post-oxygenated blood may return to the inflow side of the oxygenator 314 via a recirculation circuit. FIG. 2B illustrates that the ECMO system 300 may utilize the pump 312 to siphon (e.g., pull, drain, draw, etc.) post-oxygenated blood out of the oxygenator 314 through a tubular pathway 342 (the direction of post-oxygenated blood flowing out of the oxygenator 314 is shown by arrow 346).

As illustrated in FIG. 2B, the recirculated post-oxygenated blood may pass through the pump 312 and return to the inlet side of the oxygenator 314 via the tubular pathway 344 (the direction of post-oxygenated blood flowing out of the oxygenator 314 is shown by arrow 348). It can be appreciated the post-oxygenated blood that enters the pump 312 may combine with the deoxygenated blood being drawn from the patient through the tubular pathway 324 prior to passing by or through the semi-permeable membrane of the oxygenator 314. It is further contemplated that the post-oxygenated blood within the feedback pathway may combine with deoxygenated blood prior to entering the pump 312, within the pump 312 or within the tubular pathway 344. Accordingly, the recirculated post-oxygenated blood may increase the oxygen level in the deoxygenated blood before it passes through the semi-permeable membrane of the oxygenator 314. It can be further appreciated that as the oxygen recirculation cycle continues (e.g., as post-oxygenated blood is recirculated and mixed with deoxygenated blood prior to entering the oxygenator 314), the oxygen saturation level in the post-oxygenated blood returning to the patient will increase over time.

In the ECMO system 300, a flow regulator 380 may be positioned in the blood recirculation pathway 342 to regulate the flowrate of blood being recirculated through the pump 312 to the oxygenator 314. The flow regulator 380 may be controlled by the console 316, for example, to automatically adjust the orifice through the flow regulator 380 in response to a desired demand for adjusting the oxygen saturation level of oxygenated blood exiting the oxygenator 314 and/or returning to the patient 50. For instance, based on the oxygen saturation level of post-oxygenated blood returning to the patient, which may be sensed with an oxygen sensor as described above, the console 316 may automatically adjust (increase or decrease) the flow of blood through the flow regulator 380. In other instances, a user may manually adjust (increase or decrease) the flow of blood through the flow regulator 380 with the console or other controller.

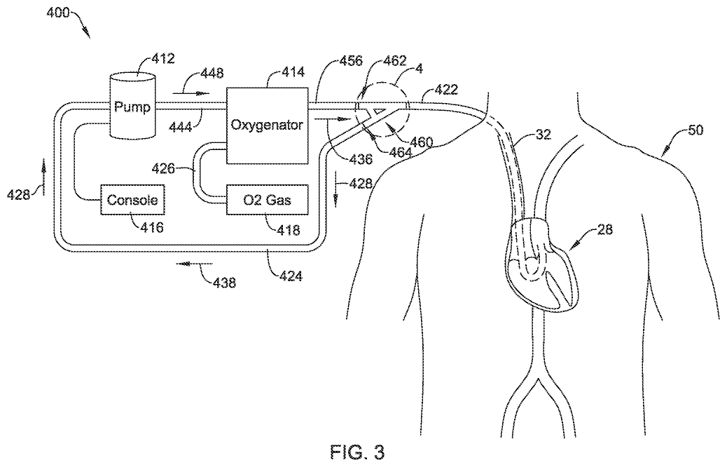

FIG. 3 illustrates another veno-venous ECMO life support system 400. The ECMO system 400 may be similar in form and function to other ECMO systems described herein. For example, the ECMO system 400 may include a blood pump 412 designed to remove deoxygenated blood from a patient 50 through a tubular blood pathway 424 (the direction of deoxygenated blood out of the patient is shown by arrow 438). It can be further appreciated that the pump 412 may also be utilized to propel blood to an oxygenator 414 through a tubular pathway 444 (the direction of deoxygenated blood flowing through the pump 412 toward the oxygenator 414 is shown by arrow 448). As the deoxygenated blood enters the oxygenator 414 it may pass by or through the semi-permeable membrane of the oxygenator 414 whereby red blood cells absorb oxygen (drawn from an oxygen source 418) and carbon dioxide is released. Further, after blood passes through the semi-permeable membrane of the oxygenator 414, the post-oxygenated blood may return to the patient (the direction of post-oxygenated blood exiting an outlet port of the oxygenator is shown by arrow 436).

Like other dual cannulation site ECMO systems described herein, the ECMO system 400 shown in FIG. 3 utilizes a dual-lumen cannula 422 introduced into the patient via the internal jugular vein 32 whereby it is further tracked into the right atrium of the heart. As described herein, the dual lumen cannula 422 may include a first, infusion lumen (e.g., inner lumen) extending coaxially within a second, drainage lumen (e.g., outer lumen). Accordingly, fluid (e.g., blood) may flow through both the infusion lumen and the drainage lumen. While not illustrated in FIG. 3, the drainage lumen may include one or more apertures disposed along its distal end region in fluid communication with the drainage lumen. Additionally, the infusion lumen may extend distally past the end of the drainage lumen. When positioned in the heart, the drainage lumen is positioned in the right atrium and infusion lumen is positioned in the main pulmonary artery, for example.

As discussed herein, the dual-lumen cannula 422 operates by returning oxygenated blood to the patient from the ECMO circuit via its infusion lumen, which may be positioned in the pulmonary artery, and withdrawing deoxygenated blood from the patient to the ECMO circuit via its drainage lumen, which may be positioned in the right atrium. FIG. 3 illustrates that the dual-lumen cannula 422 may include a hub or manifold 460 having an inlet port 462 and an outlet port 464. It can be appreciated that the inlet port 462 may be coupled to an outlet of the oxygenator 414. Accordingly, post-oxygenated blood exiting the oxygenator 414 may enter the manifold inlet port 462, whereby the post-oxygenated blood passes through the infusion lumen and into the main pulmonary artery. Further, it can be appreciated that the outlet port 464 of the manifold 460 may be coupled to the tubular blood pathway 424. Accordingly, deoxygenated blood may drain through the drainage lumen positioned in the right atrium, travel through the drainage lumen and pass into the tubular blood pathway 424, whereby the pump 412 may propel it toward the oxygenator 414. After passing through the oxygenator 414 and through the infusion lumen of the dual-lumen cannula 422 inserted into the right jugular vein 32, the post-oxygenated blood may be released in the right atrium of the patient 50.

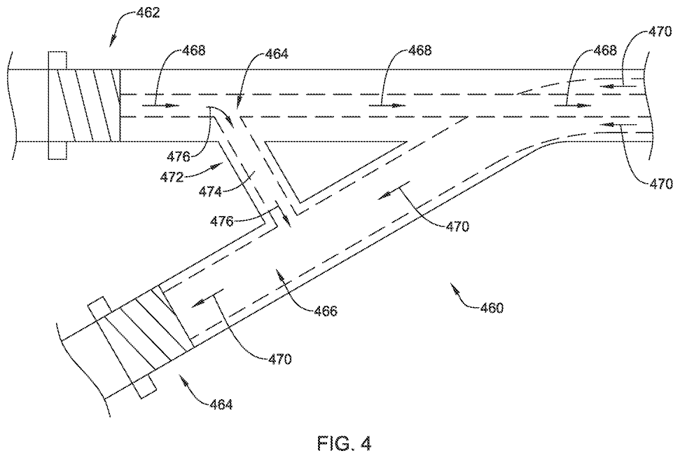

Further, the ECMO system 400 may include a recirculation or feedback circuit to recirculate blood through the oxygenator 414. As described above, after blood passes by or through the semi-permeable membrane of the oxygenator 414, some of the post-oxygenated blood may combine with deoxygenated blood being withdrawn from the patient prior to returning to the oxygenator 414. However, alternatively to the ECMO systems 10/100/200/300 described above, the ECMO system 400 utilizes a feedback pathway 472 (shown in FIG. 4) within the manifold 460 to combine the post-oxygenated blood exiting the oxygenator but not returning to the patient with deoxygenated blood being withdrawn from the patient prior to returning to the oxygenator 414.

FIG. 4 illustrates a detailed view of the manifold 460 shown in FIG. 3. The manifold 460 may include an inlet port 462 (e.g., inlet connection, luer fitting, etc.) and an outlet port 464 (e.g., outlet connection, luer fitting, etc.). As described herein, the inlet port 462 may be connected to an outlet of the oxygenator 414 via the tubular pathway 456. Additionally, the outlet port 464 may be connected to the tubular pathway 424, whereby deoxygenated blood passes from the patient 50 to the pump 412 (and oxygenator 414) via the tubular pathway 424. It can be appreciated from FIG. 4 that the manifold 460 may include a first lumen 464 designed to pass post-oxygenated blood from the inlet port 462 through the manifold 460 and into the infusion lumen of the dual-lumen cannula 422 (shown FIG. 3). For example, the arrows 468 illustrate the flow path of post-oxygenated blood passing from the oxygenator 414, through the manifold 460 and into the infusion lumen of the dual lumen cannula 422.

Additionally, FIG. 4 illustrates that the manifold 460 may include a second lumen 466 designed to pass deoxygenated blood draining through the drainage lumen (e.g., positioned in the right atrium) of the dual-lumen cannula 422 through the manifold 460 and into the tubular pathway 424 (shown FIG. 3), whereby the pump 412 may propel it toward the oxygenator 414. For example, the arrows 470 illustrate the flow path of deoxygenated blood passing from the drainage lumen of the dual-lumen cannula 422, through the manifold 460 and into the tubular pathway 424 (shown FIG. 3).

Additionally, FIG. 4 illustrates that the manifold 460 may include a feedback or recirculation pathway 472 which may include a third lumen 474 interconnecting the first lumen 464 and the second lumen 466 within the hub or manifold 460. It can be appreciated that the feedback pathway 472 is designed to permit some of the post-oxygenated blood passing through the first lumen 464 to split off and travel through the third lumen 474, whereby the post-oxygenated blood may combine with the de-oxygenated blood passing through the second lumen 466. For example, the arrows 476 illustrate the flow path of post-oxygenated blood passing from the first lumen 464 of the manifold 460, through the third lumen 474 of the feedback pathway 472 and into the second lumen 466 of the manifold 460, whereby the post-oxygenated blood may combine with the deoxygenated blood prior to passing through the tubular pathway 424 prior to passing through the semi-permeable membrane of the oxygenator 414. Although not shown, in some instances, a one-way valve may be placed along the feedback pathway 472 to allow blood to flow only in one direction through the feedback pathway 472 (e.g., only allow blood to flow through the feedback pathway 472 from the first lumen 464 (e.g., infusion lumen having oxygenated blood returning to the patient) to the second lumen 466 (e.g., drainage lumen having deoxygenated blood withdrawn from the patient).

Similar to other oxygen feedback pathways described herein, the post-oxygenated blood passing through the feedback channel 472 may increase the oxygen level in the deoxygenated blood before it passes through the semi-permeable membrane of the oxygenator 414. It can be further appreciated that as the oxygen recirculation cycle continues (e.g., as post-oxygenated blood is recirculated and mixed with deoxygenated blood prior to entering the oxygenator 414), the oxygen saturation level in the post-oxygenated blood returning to the patient will increase over time.

U.S. Pat. No. 9,168,352, entitled Dual Lumen Cannula, is herein incorporated by reference in its entirety for any and all purposes, including its disclosure of a dual lumen cannula as it relates to the dual lumen cannulas described herein.

In some embodiments, the ECMO systems described herein and/or components thereof may be made from a metal, metal alloy, polymer (some examples of which are disclosed below), a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID@ available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS polycarbonates, polyisobutylene (PIB), polyisobutylene polyurethane (PIBU), polyurethane silicone copolymers (for example, Elast-Eon® from AorTech Biomaterials or ChronoSil® from AdvanSource Biomaterials), ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is defined in the language in which the appended claims are expressed.

What is claimed is:

1. An extracorporeal blood treatment system, comprising:
a blood oxygenator having an inlet and an outlet, wherein deoxygenated blood received from a patient passes into the oxygenator inlet, wherein oxygenated blood from the oxygenator exits through the outlet and passes to the patient; and
a recirculation flow path configured to recirculate a portion of the oxygenated blood exiting the oxygenator outlet back into the oxygenator inlet.

2. The blood treatment system of claim 1, wherein the recirculated oxygenated blood combines with the deoxygenated blood prior to passing through the oxygenator.

3. The blood treatment system of claim 2, wherein the recirculated oxygenated blood combines with the deoxygenated blood within the oxygenator.

4. The blood treatment system of claim 2, wherein the recirculated oxygenated blood combines with the deoxygenated blood within the recirculation flow path.

5. The blood treatment system of claim 2, wherein the recirculated oxygenated blood has an oxygen saturation level, the deoxygenated blood has an oxygen saturation level, and wherein combining the recirculated blood with the deoxygenated blood forms partially oxygenated blood having an oxygen saturation level which is between the oxygen saturation level of the oxygenated blood and the oxygen saturation level of the deoxygenated blood prior to passing through the oxygenator.

6. The blood treatment system of claim 5, wherein the oxygen saturation of the partially oxygenated blood increases as the partially oxygenated blood passes through the oxygenator.

7. The blood treatment system of claim 5, wherein the partially oxygenated blood passing through the oxygenator has a flowrate greater than the oxygenated blood exiting the oxygenator and returning to the patient.

8. The blood treatment system of claim 5, wherein the partially oxygenated blood passing through the oxygenator has a flowrate equal to the flowrate of the blood exiting the oxygenator and returning to the patient plus the flowrate of the oxygenated blood passing into the recirculation flow path.

9. The blood treatment system of claim 1, further comprising a recirculation pump coupled to the oxygenator, wherein the recirculation pump is configured to pump the recirculated oxygenated blood back into the oxygenator.

10. The blood treatment system of claim 9, further comprising a blood pump coupled to the oxygenator, wherein the blood pump is configured to pump deoxygenated blood from the patient into the oxygenator.

11. The blood treatment system of claim 1, wherein blood passes from the oxygenator to the patient along a first blood pathway, and wherein the first blood pathway includes a first oxygen sensor positioned therein.

12. The blood treatment system of claim 11, wherein blood passes from the patient to the oxygenator along a second blood pathway, and wherein the second blood pathway includes a second oxygen sensor positioned therein.

13. The blood treatment system of claim 10, wherein the first oxygen sensor is configured to sense an oxygen saturation level of blood in the first blood pathway, the second oxygen sensor is configured to sense an oxygen saturation level of blood in the second blood pathway, and wherein the first oxygen sensor, the second oxygen sensor or both the first oxygen sensor and the second oxygen sensor are configured to send a signal to the oxygenator indicating the oxygen saturation level of blood in the first blood pathway and the second blood pathway, respectively.

14. The blood treatment system of claim 13, wherein the oxygenator is configurated to adjust the oxygen saturation level of blood in the first blood pathway in response to a signal received from the first oxygen sensor, the second oxygen sensor or both the first oxygen sensor and the second oxygen sensor.

15. The blood treatment system of claim 1, further comprising a dual lumen cannula coupled to the oxygenator, the dual lumen cannula including a manifold having a first blood pathway, a second blood pathway and a third blood pathway, and wherein the third blood pathway connects the first blood pathway to the second blood pathway.

16. The blood treatment system of claim 15, wherein the oxygenated blood passes from the oxygenator to the patient through the first blood pathway of the manifold, wherein deoxygenated blood passes from the patient to the oxygenator through the second blood pathway of the manifold, and wherein a portion of the oxygenated blood passes from the first blood pathway, through the third blood pathway and combines with deoxygenated blood in the second blood pathway.

17. An extracorporeal blood treatment system, comprising:
a blood circulation pathway coupled to a blood oxygenator, wherein the blood circulation pathway is configured to pass deoxygenated blood withdrawn from a patient through a blood oxygenator and back to the patient; and
a blood recirculation pathway, the blood recirculation pathway configured to recirculate oxygenated blood exiting the oxygenator back into the oxygenator prior to returning to the patient.

18. The blood treatment system of claim 17, wherein the blood passing through oxygenator has a flowrate greater than the oxygenated blood exiting the oxygenator and passing to the patient.

19. The blood treatment system of claim 17, wherein the blood passing through the oxygenator has a flowrate equal to the flowrate of the oxygenated blood exiting the oxygenator and passing to the patient plus the flowrate of oxygenated blood exiting the oxygenator and passing into the blood recirculation pathway.

20. An extracorporeal blood treatment system, comprising:
a blood oxygenator having an inlet and an outlet; and
a dual-lumen cannula coupled to the oxygenator, the cannula having a distal end configured to be positioned in a patient and a proximal end including a manifold, wherein the manifold includes a first blood pathway in fluid communication with the oxygenator outlet, a second blood pathway in fluid communication with the oxygenator inlet, and a third blood pathway connecting the first blood pathway to the second blood pathway;
wherein the manifold is configured to pass oxygenated blood received from the oxygenator through the first blood pathway;
wherein the manifold is configured to pass deoxygenated blood received from the patient through the second blood pathway;
wherein the manifold is configured to pass a portion of the oxygenated blood from the first blood pathway through the third blood pathway such that it combines with deoxygenated blood in the second blood pathway.
